(19) United States
(12) Patent Application Publication
(10) Pub. No.: US 2007/0185584 A1
(43) Pub. Date: Aug. 9, 2007

(54) SMALL JOINT HEMIARTHROPLASTY

(76) Inventors: Robert A. Kaufmann, Pittsburgh, PA (US); Robert Goitz, Gibsonia, PA (US)

Correspondence Address: REED SMITH LLP, P.O. BOX 488, PITTSBURGH, PA, 15230-0488, US

(21) Appl. No.: 11/670,578

(22) Filed: Feb. 2, 2007

Related U.S. Application Data
(60) Provisional application No. 60/764,851, filed on Feb. 2, 2006.

(51) Int. Cl. A61F 2/42

(52) U.S. Cl. 623/21.15

(57) ABSTRACT

Methods and apparatuses for digit joint arthroplasty. The present invention preferably allows for the treatment of disorders of digit joints generally and interphalageal joints more specifically. The present invention provides implants for the replacement of digit joint cartilage. The implants of the present invention preferably include a head and a shaft. The head may be shaped similarly to the cartilage that is being replaced. The shaft is adapted so as to be able to be fit into, for example, the phalanx. In certain preferred embodiments, the shaft is threaded so that it may gain purchase to the phalangeal cortex. The articulating portion of the implant preferably mimics the articular surface of the native phalanx and thereby places minimal motion restriction on the patient. The implants and methods of the present invention have particular utility with the DIP joint of the hand.

### CROSS-REFERENCE TO RELATED APPLICATION

This application claims the benefit under 35 U.S.C. §119(e) of the earlier filing date of U.S. Provisional Application Ser. No. 60/764,851 filed on Feb. 2, 2006.

### BACKGROUND OF THE INVENTION

1. Field of the Invention

The present invention relates to methods and apparatuses for the treatment of disorders of the joints (e.g., osteoarthritis) of digits generally, of the joints of fingers specifically, and of the joints of the distal interphalangeal joint (DIP) more specifically.

2. Description of the Background

There are three bones in each finger that are termed the proximal phalanx, the middle phalanx, and the distal phalanx. Each finger also has three joints.

The first joint is where the finger articulates with the hand. Within this first joint, one of the bones within the palm of the hand (a metacarpal) joins with the first bone of the finger (a proximal phalanx). The second joint is where the proximal phalanx articulates with the middle phalanx and is termed the proximal interphalangeal (PIP) joint. The most distal joint of the finger is called the distal interphalangeal (DIP) joint. Each of these joints is covered with articular cartilage. The cartilage allows the bones to slide easily relative to one another as the joint moves through its range of motion. Motion at the DIP joint is both rotational and translational.

Degenerative joint diseases such as osteoarthritis commonly affect the DIP joints and cause articular degeneration and marginal bone spur formation. DIP joint arthritis is often painful and may interfere with function of a single finger or of the entire hand. When conservative measures are no longer capable of controlling symptoms, operative intervention may be recommended.

Arthodesis (i.e., joint fusion) is the procedure of choice for end stage arthritis but results in complete loss of joint motion. Patient demands or specialized functional requirements may necessitate a solution that maintains joint motion.

Finger joint arthroplasty through replacement of half of the joint (hemiarthroplasty) has been described using a vitallium metacarpal head replacement (Burman. “Vitallium Cup Arthroplasty of Metacarpophalangeal and Interphalangeal joints of fingers.” Bull. Hosp. Joint Dis. 1940; 1:79-89). This research did not address the DIP joint and the apparatuses described therein did not re-establish an adequate range of motion.

Replacing both sides of a finger joint has been disclosed in several prior art patents using various implants to accomplish this goal. For example, U.S. Pat. No. 4,246,662 discloses a single-piece silicone implant that spans the finger joint. It has an enlarged central portion with two stems for insertion into the medullary canal of the bones on either side of the joint. That patent only addresses treatment of the metacarpophalangeal joint and the proximal interphalangeal joint, but not the DIP joint. The joint prosthesis of U.S. Pat. No. 4,246,662 patent alleges improvement over other metacarpophalangeal joint prostheses through improved force distribution onto the metacarpal and improved resistance to breakage. Specifically, it is an improvement over U.S. Pat. No. 3,462,765, which was A. B. Swanson's original silicone arthroplasty for the metacarpo-phalangeal (MCP) and proximal interphalangeal (PIP) joints. U.S. Pat. No. 4,246,662 is similar to the past efforts except for an offset hinge. These cited patents, and others in the art, employ a single piece of silicone which has been shown to deteriorate and break over time.

Other patents such as U.S. Pat. No. 3,681,786 are directed to small joint replacement. The devices disclosed therein bear similarities to other prior art devices, but instead makes use of a single piece of inherently flexible elastomeric material and a polyester felt pad embedded within the prosthesis to provide for ingrowth of body tissue.

U.S. Pat. No. 5,425,777 discloses a method of replacing articular surfaces on both sides of a finger joint through insertion of prosthetic devices into adjacent bones. That patent does not specifically refer to its use in the DIP joint, but such an approach would be problematic since the distal phalanx is unlikely to accept such an implant because of its small size. Even if the distal phalanx was able to incorporate this implant, the distal phalanx would be prone to bony failure as a consequence of the substantial bone resection that would accompany implant insertion. The distal phalanx with a medullary canal diameter of 3 to 4 millimeters is not able to accommodate designs that require fixation in or partial resection of the distal phalanx.

U.S. Pat. No. 5,458,647 discloses a hinge-like implant for use in finger joint replacement. The implants disclosed therein require insertion of an implant into the medullary canal of the distal phalanx and securing that implant with cement which would also be accompanied by significant bone resection in order to accommodate both the distal phalanx component and cement mantle. As already mentioned, the distal phalanx is too small to incorporate the stem required in this hinged implant invention. That design also mandates that the finger flex and extend along a fixed axis of rotation without allowing for translation to occur. Such a motion is not physiologic because it does not reproduce the actual kinematics of the normal DIP joint, which moves through both sliding as well as pure rotation.

U.S. Pat. Nos. 5,782,927 and 6,159,247 specifically address the treatment of the metacarpal-phalangeal joint, but not the DIP joint. This implant consists of non cemented “poly carbon” stemmed components that are congruent with the joint that it is replacing. An effort is made to reproduce normal kinematics through close matching of the implant to the size and morphology of the native joint. Wear between mating articulating surfaces is minimized through a design that allows biological fluids to lubricate their motion.

U.S. Pat. No. 6,699,292 discloses a prosthesis for the PIP joint that is similar to the design listed above for the MCP joint. The invention disclosed, if used in the distal phalanx, would be accompanied by the difficulty of removing bone from the distal phalanx in order to allow implant insertion.

U.S. Pat. No. 6,475,242 discloses threaded implants for use with DIP, PIP or MCP joints. It specifically discloses an artificial joint assembly that includes externally threaded stems that are both threaded into adjacent phalanges. The two assemblies are connected through flexible ‘U-shaped’ member that allows hinged flexion. That implementation has the disadvantage of creating non-anatomic hinge joint with the drawbacks outlined previously. In addition, insertion of any implant into the distal phalanx would be accompanied by all of the difficulties disclosed hereinabove.

U.S. Pat. No. 6,811,568 discloses an artificial joint that includes two parts. That invention relates to an artificial MCP joint or elbow joint. The patent requires the insertion of two implants, one into the distal bone and one into the proximal bone with a stem and socket, which would be accompanied by the difficulties discussed hereinabove.

Thus, there has been a long-standing need for an apparatus that replaces the joints of phalanges in general that can be accommodated by the small bones adjacent to this particular joint. In addition, the prior art has felt a long-standing need for prostheses that gain fixation into small bones of the phalanges which and stably maintain their placement and function. Preferably, the implant would mimic the natural cartilage and thereby reduce stress and wear on the prosthesis. The present invention satisfies these long-felt needs.

### BRIEF DESCRIPTION OF THE DRAWINGS

For the present invention to be clearly understood and readily practiced, the present invention will be described in conjunction with the following figures, wherein like reference characters designate the same or similar elements, which figures are incorporated into and constitute a part of the specification, wherein:

FIG. 1 displays the anatomy of the DIP joint of the finger; and

FIG. 2 shows an implant of the present invention positioned in the DIP joint.

### SUMMARY OF THE INVENTION

The present invention is directed generally to methods and apparatuses for the replacement of the joints of digits generally and of phalanges more particularly. The present invention further encompasses methods and apparatuses for performing hemi- or full arthroplasty of joints of the digits. In particularly-preferred embodiments, the present invention is directed to methods and apparatuses for performing hemiarthroplasty of the DIP joint. However, the present invention is useful in not only the DIP joint, but also the other joints of fingers and toes. Preferably, the implants of the present invention gain fixation into small bones of the phalanges, which through the high cortical to cancellous bone ratio, are sturdy enough to accommodate the implant insert. Preferred implants of the present invention employ a threaded stem to gain fixation into the bones of the phalanges. The implants of the present invention may be used singly to perform hemiarthoplasty or in pairs at the same joint to perform full arthorplasty of that joint.

In particular, presently-preferred embodiments of the present invention are directed to the replacement of the proximal aspect of the DIP joint with an implant that functionally replaces any lost or damaged joint cartilage. In a presently-preferred embodiment, the middle phalanx is drilled and tapped to create a threaded intramedullary canal that then provides for stable fixation of implants of the present invention. Of particular utility, the present procedures and apparatuses can be used in lieu of arthrodesis. The methods of the present invention may be performed in a relatively safe manner without violating the dorsal extensor mechanism or the dorsal skin. The presently-preferred methods have the advantage of maintaining both the extensor and flexor insertions on the distal phalanx while at the same time allowing removal of osteophytes on the distal phalanx that are often unsightly and painful to the patient.

### DETAILED DESCRIPTION OF THE INVENTION

It is to be understood that the figures and descriptions of the present invention have been simplified to illustrate elements that are relevant for a clear understanding of the invention, while eliminating, for purposes of clarity, other elements that may be well known. The detailed description will be provided hereinbelow with reference to the attached drawings.

The present invention is directed generally to methods and apparatuses for the treatment of disorders of the joints such as osteoarthritis. The present invention further encompasses methods and apparatuses for performing replacement of portions of joints or entire joints. In general, the present invention is useful in replacing joints formed between small bones (e.g., the fingers and toes). For example, the present invention has particular utility in the treatment of joints of the digits generally and of the phalanges more particularly.

While the present invention is described with particular reference to the joints of the fingers and even more particularly with respect to the DIP joint, it will be understood by those of skill in the art that the present invention is generally applicable to joints and particularly to joints abutted by small bones such as those present in the toes.

In particularly-preferred embodiments, the present invention is directed to methods and apparatuses for performing hemiarthroplasty of the DIP joint.

Preferably, the implants of the present invention gain fixation into small bones of the phalanges which, through the high cortical to cancellous bone ratio, are sturdy enough to accommodate the implant insert. Preferred implants of the present invention employ a threaded stem to gain fixation into the bones of the phalanges. The implants of the present invention may be used singly to perform hemiarthroplasty or in pairs at the same joint to perform full arthroplasty of that joint.

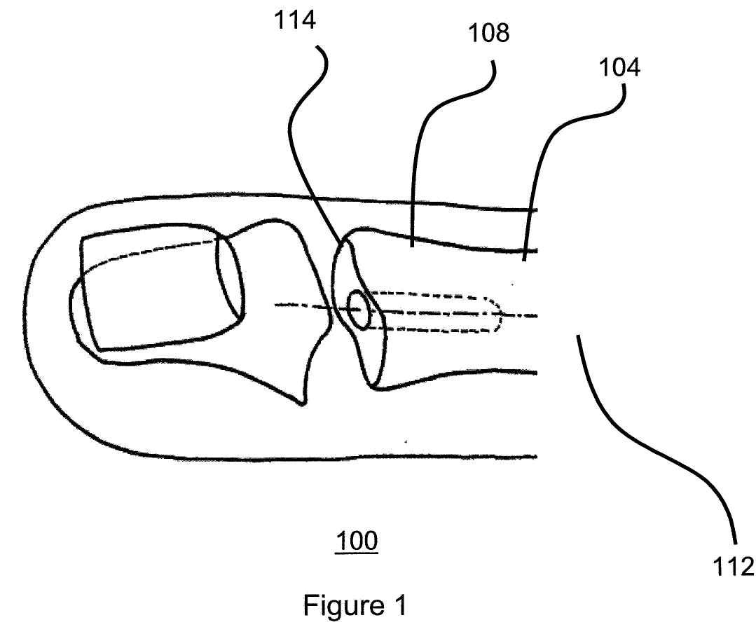

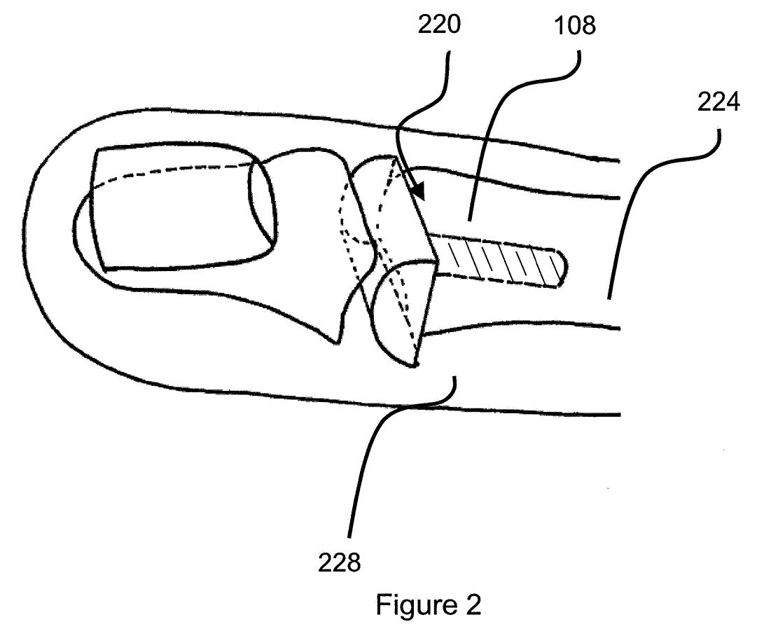

The present invention encompasses a middle phalanx hemiarthroplasty that has not been previously described for the DIP joint. The general anatomical structure of the DIP joint 100 may be observed in FIG. 1. The present invention includes resurfacing of the distal aspect 108 of the middle phalanx 104 by using an implant 220 (see FIG. 2) that approximates the articular surface that it is replacing and, therefore, restores a similar range of motion to the joint. The implant portion that resurfaces the joint is rigidly fixed into the middle phalanx intramedullary canal 112 of the middle phalanx 104 through a threaded shaft 224. A joint surface-restoring implant 220 that acts as a hemiarthroplasty (i.e., where only one side of the joint is resurfaced) represents a novel approach to the management of DIP joint degeneration.

The implants of the present invention are preferably designed to provide for joint stability. Stability of a joint, defined as the resistance to subluxation under physiologic stresses, is normally the result of the mechanical interaction of the articular contours, the dynamic support of the investing musclotendinous units, and the static viscoelastic constraint of the capsuloligatmentous structures. The present inventive design restores the articular contour of the middle phalanx portion of the DIP joint while maintaining the dynamic support of the extensor and flexor tendons. The methods of the present invention result in the initial disruption, but subsequent repair of the collateral ligaments (proper and accessory) on one side of the DIP joint. The present hemiarthroplasty design is preferably modular and may be sized to prevent over-stuffing and over-constraining the joint. Those attributes are advantageous to reproducing normal physiologic motion and limiting the stresses transmitted through the prosthesis to the stem-bone interface.

The surgical methods of the present invention begin with a longitudinal incision between the conjoined lateral bands (extensor mechanism) dorsally and the proper collateral ligament volarly. The joint capsule is preferably incised dorsally but the volar plate, extensor, and flexor tendons are maintained. The proper and accessory collateral ligaments are reflected off of their origin on the middle phalanx. The head of the middle phalanx is removed with a ronguer or oscillating saw. The joint is then jawed open to expose the intramedullary canal of the middle phalanx to allow for drilling and tapping. The present approach preferably does not violate the dorsal extensor mechanism or flexor tendon insertion and keeps those important dynamic stabilizers intact.

The present invention preferably disturbs soft tissue structures as little as possible. In preferred embodiments, only the collateral ligaments are reflected off of their origin and are later repaired once the implant is seated. Collateral ligaments are complex structures whose individual fascicles are under differential tension and whose properties depend on joint position and load. The collateral and accessory collateral ligaments are responsible for resisting forces that may sublux this joint. When they are detached during joint hemiarthroplasty, their resistance to subluxation is compromised.

Restoration of the collateral ligamentous system through reattachment to the native site of origin is a presently-preferred step in the methods of the present invention.

The implants of the present invention address several outstanding concerns of the art. Firstly, the implant preferably restores a functional range of motion to the joint. That goal is accomplished by replacing the distal articular portion of the middle phalanx. The articulating portion will preferably be shaped to match the normal contour of the middle phalanx that is to be replaced. The present design thus preferably achieves proper fixation of the articulating portion to the middle phalanx.

Through restoration of normal anatomy, the present invention maintains a mechanical advantage of the flexor and extensor tendons similar to what occurs in normal joints. Specifically, in order for a joint to achieve normal motion, the tendons must pass the joint at the proper anatomic distance from its center of rotation. Accurate reproduction of the physiologic center of rotation mandates design of an implant that reproduces native anatomy.

The articulating portion of the implants of the present invention are preferably shaped in a manner that is similar to a cam in that the anteroposterior axis is longer than the distoproximal axis. By replacing the articular surface with a similar shaped metal surface, the present invention maintains the joint's inherent mechanical advantage. As discussed hereinabove, that mechanical advantage is lost in prior art designs that use a hinge joint with a constant center of rotation. In other preferred embodiments, the head of the implant has a half-round or semi-circular profile. One of skill in the art will recognize variations of the particular shape of the arthroplasty that will be functional within the context of the present invention. The head is preferably attached to a rod that is inserted into the intramedullary canal of the middle phalanx.

The design of the present invention also includes considerations of the prosthetic size that will make the inventive implants compatible with the joint size. The relationship between the intramedullary shaft and the articular surface will be based on cadaver measurements as well as computerized axial tomography (CT) scans of the joint. It is expected that multiple implant sizes will accommodate various finger dimensions.

The design of the present invention also preferably ensures firm stem seating to resist shearing, bending and rotational stresses that may be encountered after implantation. Firm seating may be accomplished through a variety of methods such as press-fitting the stem into the intramedullary canal within the middle phalanx or cementing the implant into the intramedullary canal. In a particularly-preferred embodiment, the middle phalanx is drilled and tapped. After this is accomplished, the implant whose stem is a threaded rod may be screwed into the middle phalanx bone.

Since bone cement is not required for implant fixation into the middle phalanx in preferred embodiments, removal of the implant may be performed with ease as required, such as in cases of infection. Also, due to the relatively small amount of middle phalanx bone that is removed, an arthrodesis either for continued pain or infection may easily be performed at a later time.

The present invention further provides for easy accommodation of differences in joint size. This goal is accomplished through providing multiple drill sizes with matching taps for each implant stem size. In the embodiments with a threaded rod 224, the middle phalanx articular surface 108 as displayed in FIG. 2 is preferably removed with a hand held instrument (e.g., a rongeur) and then the intramedullary canal 112 is preferably drilled with a small drill bit. If the drill bit does not engage cortical bone, then a larger drill bit would preferably be used until the middle phalanx 104 is ready for tapping. The implants 220 of the present invention also preferably include a head 228 that mimics the curvature of the natural cartilage. The implant 220 shown in FIG. 2 displays a head 228 with a semi-cylindrical design, though other designs may be implemented that more accurately approximately the natural cartilage. The tap that is used will preferably correspond to the drill size that was needed to gain cortical purchase. Once the middle phalanx 104 is tapped, the threaded rod 224 is threaded into place and the attached condyle seated onto the distal phalanx 114, as displayed in FIG. 2.

The implants of the present invention may be formulated of any physiologically-compatible material. Suitable materials include titanium, cobalt, stainless steel, silicone, and physiologically-compatible plastics. Currently in orthopedics, there are three principal metal alloys used in joint replacement technology: titanium, cobalt, and stainless steel. Those three metals possess desirable mechanical properties such as high tensile strength and corrosion resistance. Newly-engineered plastics are regularly emerging and those with suitable biocompatibility and engineering characteristics will be recognized by those of skill in the art to be useful within the context of the present invention.

Factors determining arthroplasty survivability include prosthetic wear, formation of wear debris, and tissue reaction to the wear debris. Particulate debris generated by wear between two man-made surfaces such as metal and polyethylene (such as used in total knee replacement arthroplasty) will not occur in the present design since only one surface of the joint is replaced. An inflammatory reaction that is caused by wear debris can therefore not take place. The present design leaves the proximal portion of the distal phalanx intact with the exception of osteophytes that may need removal. Since the distal phalanx is not resurfaced, it is expected that some continued erosion of this portion may occur over time. This progressive local bone loss should not threaten the function of the implants of the present invention.

The present invention also addresses adequate material wear and strength characteristics. A material to be considered for the present application is titanium which is strong, durable, and possesses a high resistance to corrosion as well as low thermal conductivity. A consideration for ensuring long term durability of the present implant is the osseointegration of the stem. Bone ingrowth fixation using sintered metal, plasma spray, porous polyethylene, or hydroxyapatite stem coating has not proven to be effective in small hand bones (Friedman, Black, Galante, Jacobs, and Skinner. “Current concepts in orthopaedic biomaterials and implant fixation.” Instr. Course Lect. 1994; 43:233-255). The use of pure titanium screws for permanent fixation (Branemark, Hansson, Adell et al. “Osseointegrated implants in the treatment of the endentulous jaw. Experience from a 10-year period.” Scand. J. Plast. Reconstr. Surg. Suppl. 1977; 16:1-132) has proven effective in dental reconstruction and is showing encouraging results in stem fixation for PIP joint replacement (Lundborg, Branemark, and Carlsson. “Metacarpophalangeal joint arthroplasty based on the osseointegration concept.” J. Hand Surg. 1993; 18b:693-703). The implants of the present invention may further be employed with pharmaceutical and biotechnological agents promote the osseointegration of the step of the implant. One of skill in the art will recognize multiple pharmaceutical compounds with utility in the present invention.

While the present invention was described hereinabove for use with a threaded stem, the implant could be fixed to the phalangeal bone by a variety of mechanisms. For example, the stem of the implant may be secured with a press fit, cemented in place, or even screwed into the bone. Alternatively, the implants of the present invention may include stems that are irregularly shaped in a manner that secures them firmly in the bone.

Those of skill in the art will recognize that numerous modifications of the above-described methods and apparatuses can be performed without departing from the present invention. For example, one of skill in the art will recognize that the specific design of the head of the implant or the specific manner of securing of the implant may be varied according to well known practices.

We claim:

1. An implant adapted for use in arthroplasty of a digit joint, comprising
a head; and
a shaft, wherein said shaft is adapted to fit into an intramedullary hole of a phalanx.

2. The implant of claim 1, wherein said digit joint is an interphalangeal joint.

3. The implant of claim 2, wherein said interphalangeal joint is the proximal interphalangeal joint.

4. The implant of claim 2, wherein said interphalangeal joint is the distal interphalangeal joint.

5. The implant of claim 4, wherein said phalanx is the middle phalanx.

6. The implant of claim 5, wherein said intramedullary hole is in a distal aspect of said middle phalanx.

7. The implant of claim 4, wherein said shaft is adapted to be secured into said intramedullary hole.

8. The implant of claim 7, wherein said shaft is threaded.

9. The implant of claim 7, wherein said intramedullary hole in the distal aspect of said phalanx has been drilled and tapped.

10. The implant of claim 7, wherein said implant is adapted to be screwed into said intramedullary hole.

11. The implant of claim 1, wherein said head is contoured to approximate the shape of articular cartilage.

12. The implant of claim 1, wherein said head is semi-circular.

13. The implant of claim 1, wherein said implant is made of a material selected from the group consisting of titanium, cobalt, stainless steel, silicone, and engineered plastics.

14. A method of performing arthroplasty of a digit joint, comprising the steps of:
drilling into a phalanx to create a intramedullary canal;
inserting an implant into said intramedullary canal.

15. The method of claim 14, wherein said implant includes a head that is contoured to approximate the shape of articular cartilage and a shaft.

16. The method of claim 14, further comprising tapping said intramedullary canal, after said drilling step.

17. The method of claim 15, wherein said shaft is threaded.

18. The method of claim 14, wherein said digit joint is the distal interphalangeal joint.

19. The method of claim 18, wherein said phalanx is the middle phalanx.

20. The method of claim 19, wherein said intramedullary canal is in a distal aspect of said middle phalanx.

21. The method of claim 20, wherein said method maintains the extensor and flexor insertions on the distal phalanx.
